(19) United States
(12) Patent Application Publication
(10) Pub. No.: US 2017/0182363 A1
(43) Pub. Date: Jun. 29, 2017

(54) Exercise Health Management Method, Terminal and System

(71) Applicant: Xiamen Cowell Industrial Ltd., Xiamen City (CN)

(72) Inventor: Yao-Jun Li, Xiamen City (CN)

(73) Assignee: Xiamen Cowell Industrial Ltd.

(21) Appl. No.: 15/390,926

(22) Filed: Dec. 27, 2016

(30) Foreign Application Priority Data
Dec. 28, 2015 (CN) 2015-11002361.0
Jan. 29, 2016 (CN) 2016-10064246.4

(51) Int. Cl. A63B 24/00; A61B 5/01; A61B 5/021; A61B 5/024; A61B 5/00

(52) U.S. Cl. CPC A63B 24/0075; A61B 5/02438; A61B 5/6823; A61B 5/021; A61B 5/01; A63B 2230/062; A63B 2230/305; A63B 2230/505; A63B 2230/015

(57) ABSTRACT

Herein is described an exercise health management method, terminal and system. The method includes receiving original data collected by at least one data collecting terminal; generating an exercise solution based on the original data; and transmitting the exercise solution to exercise equipment corresponding to the exercise solution. The exercise health management method, terminal and system can accomplish the function of a virtual personal coach and may synchronize user data to a cloud service so that the user may receive professional exercise guidance at home. The exercise planning and reminding mode encourages the user to continue their initiative to exercise. Also, sharing exercise outcomes makes the exercise more rewarding and attracts more people to participate. Exercise amount is collected in all weathers and analyzed for the purpose of continuous exercise management.

### CROSS-REFERENCE TO RELATED APPLICATION

This application claims priority from China Patent Application Nos. 201511002361.0 and 201610064246.4, filed on Dec. 28, 2015 and Jan. 29, 2016 respectively, at the State Intellectual Property Office of the P.R.C., the content of which is hereby incorporated by reference in its entirety for all purposes.

### BACKGROUND OF THE INVENTION

1. Field of the Invention

The present invention relates to the communication technology field, and particularly to an exercise health management method, terminal and system.

2. Description of the Related Art

Prior art products are stand-alone machines and user data can not be synchronized. Professional guidance is not available in the case where exercise is performed incorrectly in the use of the products. Sport modalities are monotonous without social comparison, resulting in poor exercise initiative.

### SUMMARY OF THE INVENTION

In view of the above-mentioned technical problems, the present invention provides an exercise health management method, terminal and system that can avoid user injuries due to excessive or unguided exercise.

In accordance with an aspect of the present invention, provided is an exercise health management method which includes the steps of: receiving original data collected by at least one data collecting terminal; generating an exercise solution based on the original data; and transmitting the exercise solution to exercise equipment corresponding to the exercise solution.

Furthermore, the step of generating an exercise solution based on the original data may include: calculating a comprehensive exercise capability and qualified exercise objective based on the original data; and generating an exercise solution according to the comprehensive exercise capability and qualified exercise objective.

The original data collected by the at least one data collecting terminal includes, but is not limited to, pace counting data collected by an intelligent watch, heart rate data collected by an intelligent chest strap, blood pressure data collected by an intelligent blood pressure monitor, water intake amount data collected by an intelligent cup, body temperature data collected by an intelligent thermometer and/or weight data collected by an intelligent body weight scale.

Furthermore, the method may further include: storing the original data and/or storing the exercise solution.

Furthermore, after the step of transmitting the exercise solution to exercise equipment corresponding to the exercise solution, the method may further include: receiving original data collected by the at least one data collecting terminal in real time; and adjusting in real time the exercise solution based on said original data.

In accordance with another aspect of the present invention, further provided is an exercise health management terminal which includes a first receiving module, a generating module and a transmitting module. The first receiving module is configured to receive original data collected by at least one data collecting terminal. The generating module is configured to generate an exercise solution based on the original data received by the first receiving module. The transmitting module is configured to transmit the exercise solution to exercise equipment corresponding to the exercise solution.

Furthermore, the generating module may include a calculating unit and a generating unit. The calculating unit is configured to calculate the comprehensive exercise capability and qualified exercise objective based on the original data received by the first receiving module. The generating unit is configured to generate an exercise solution according to the comprehensive exercise capability and qualified exercise objective.

Furthermore, the exercise health management terminal may further include a server. The server is configured to store the original data received by the first receiving module and/or store the exercise solution generated by the generating module.

Furthermore, the exercise health management terminal may further include a second receiving module and an adjusting module. The second receiving module is configured to receive in real time the original data collected by the at least one data collecting terminal. The adjusting module is configured to adjust the exercise solution based on the original data.

In accordance with another aspect of the present invention, further provided is an exercise health management system which includes the described exercise health management terminal.

The exercise health management method, terminal and system can accomplish the function of a virtual personal coach and may synchronize user data to a cloud service so that the user may receive professional exercise guidance at home. The exercise planning and reminding mode encourages the user to continue their initiative to exercise. Sharing exercise outcomes, makes exercise using the present invention more rewarding and attracts more people to participate in the exercise. Exercise amount is collected in all weathers and analyzed for the purpose of continuous exercise management.

### BRIEF DESCRIPTION OF THE DRAWINGS

FIG. 1 is a flow chart showing an exercise health management method according to embodiment I of the present invention.

FIG. 2 is a block diagram showing an exercise health management terminal according to embodiment II of the present invention.

### DESCRIPTION OF THE PREFERRED EMBODIMENTS

Illustrative embodiments of the present invention will be described in detail below with respect to the figures.

### Embodiment I

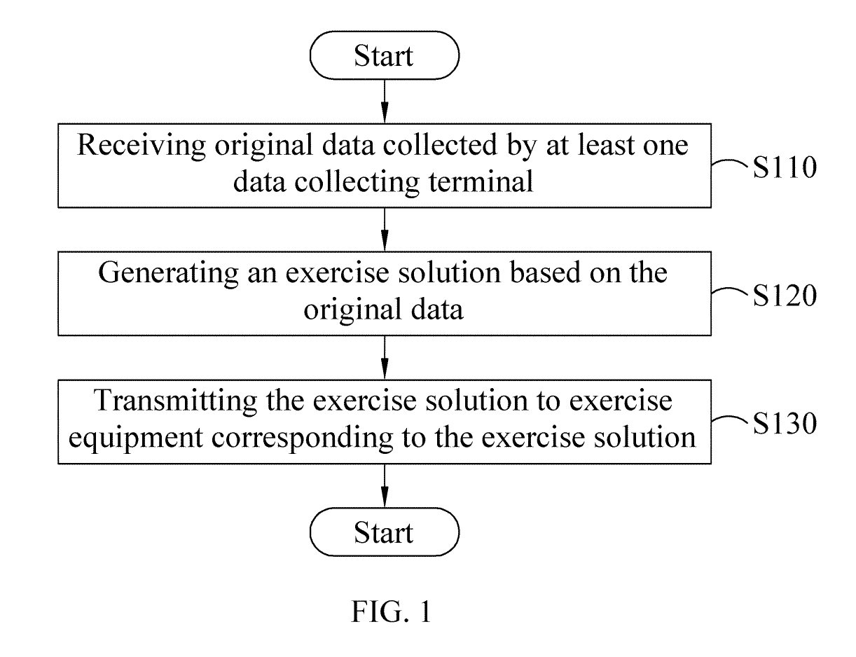

The following refers to FIG. 1, which is a flow chart showing an exercise health management method according to embodiment I of the present invention. The embodiment provides an exercise health management method which includes the following steps.

A step S110 of receiving original data collected by at least one data collecting terminal. The original data collected by the at least one data collecting terminal may be received via a Bluetooth interface or other wireless interface. The original data collected by the at least one data collecting terminal includes, but is not limited to, pace counting data collected by an intelligent watch, heart rate data collected by an intelligent chest strap, blood pressure data collected by an intelligent blood pressure monitor, water intake amount data collected by an intelligent cup, body temperature data collected by an intelligent thermometer and/or weight data collected by an intelligent body weight scale. The original data is stored for synchronization with a cloud service of all the received original data.

A step S120 of generating an exercise solution based on the original data, which may include the steps of calculating a comprehensive exercise capability and qualified exercise objective based on the synchronized original data, and generating an exercise solution according to the comprehensive exercise capability and qualified exercise objective. Then the exercise solution may be stored.

A step S130 of transmitting the exercise solution to exercise equipment corresponding to the exercise solution. The exercise solution may be customized to the specific exercise equipment and transmitted to the corresponding exercise equipment via a wireless interface for automatic execution upon starting the exercise equipment.

After transmitting the exercise solution to the applicable exercise equipment, the method may further include returning to step S110 of receiving the original data collected by the at least one data collecting terminal, then analyzing the exercise habit and capability of the user, and then adjusting the exercise solution based on the original data, all done in real time.

The technical solution of the present invention is illustrated in an example showing how to apply the method provided in embodiment I in a specific application scenario and is described as follows. User A collects original data of themselves with an intelligent watch, an intelligent chest strap, an intelligent blood pressure monitor, an intelligent thermometer and an intelligent body weight scale, these being the at least one data collecting terminal, and exercises on a running machine (the exercise equipment corresponding to the exercise solution). In this example, the method includes the following steps.

User A collects pace counting data with the intelligent watch, heart rate data with the intelligent chest strap, blood pressure data with the intelligent blood pressure monitor, water intake amount data with the intelligent cup, body temperature data with the intelligent thermometer and weight data with the intelligent body weight scale, and then transmits all the above data to an exercise health management terminal. The exercise health management terminal uploads the data to a server via a Bluetooth module in order to store the data. The server stores a plurality of items, which are exercise indexes and scores corresponding to the received original data information. An index item is recommended and selected according to the score. Each of the index items represents different levels of difficulty and amount of exercise. The cloud service of the server synchronizes user data and provides inquiry service at the same time.

When user A wants to exercise, they download the synchronized original data from the server with the exercise health management terminal. The exercise health management terminal calculates the comprehensive exercise capability and qualified exercise objective for user A with said synchronized original data; and then generates an exercise solution according to said comprehensive exercise capability and qualified exercise objective, such as a running period of three days with an exercise duration of two hours etc. User A then transmits the exercise solution to the running machine via a Bluetooth module. The running machine receives the exercise solution and automatically locks the operation panel. After the user turns on the machine, it automatically runs the exercise solution and the user performs effective exercise by accomplishing the set exercise solution with the running machine.

In the exercise process, the at least one data collecting terminal monitors the health of user A, uploads the data in real time, and adjusts the exercise solution in real time to avoid potential injury to the user due to excessive or unguided exercise.

The generated exercise solution sets an exercise objective for the user, and reminds the user of the exercise push to share function. The at least one data collecting terminal monitors exercise at all times. The user can share the exercise outcomes via social software such as Wechat, QQ, Facebook and Twitter from the intelligent exercise health management terminal.

The exercise health management method provided can accomplish the function of a virtual personal coach and may synchronize user data to a cloud service so that the user may receive professional exercise guidance at home. The exercise planning and reminding mode encourages the user to continue their initiative to exercise. Also, sharing exercise outcomes makes the exercise using the present invention more rewarding and attracts more people to participate in the exercise. Exercise amount is collected in all weathers and analyzed for the purpose of continuous exercise management.

### Embodiment II

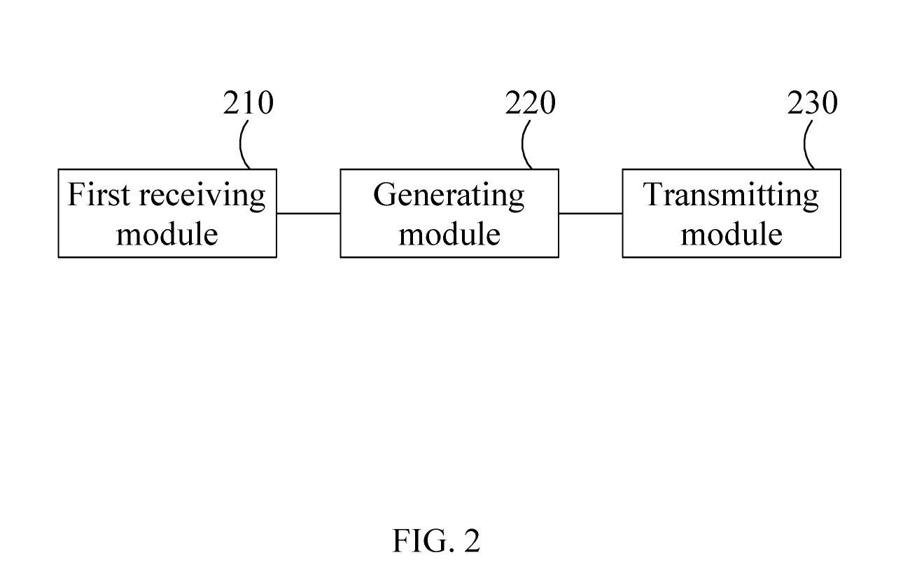

The following refers to FIG. 2, which is a block diagram showing an exercise health management terminal according to embodiment II of the present invention. The exercise health management terminal provided in the embodiment of the present invention includes a first receiving module 210, a generating module 220 and a transmitting module 230. The first receiving module 210 is configured to receive original data collected by at least one data collecting terminal. The generating module 220 is configured to generate an exercise solution based on the original data received by the first receiving module, and may include a calculating unit and a generating unit. The calculating unit is configured to calculate a comprehensive exercise capability and qualified exercise objective based on the original data received by the first receiving module 210. The generating unit is configured to generate an exercise solution according to the comprehensive exercise capability and qualified exercise objective. The transmitting module 230 is configured to transmit the exercise solution to exercise equipment corresponding to the exercise solution.

The exercise health management terminal may further include a server configured to store the original data received by the first receiving module. The server may store the exercise solution generated by the generating module and synchronize the original data to a cloud service.

Furthermore, the exercise health management terminal may further include a second receiving module and an adjusting module. The second receiving module is configured to receive in real time the original data collected by the at least one data collecting terminal. The adjusting module is configured to adjust in real time the exercise solution based on the original data.

The exercise health management terminal provided can accomplish the function of a virtual personal coach and may synchronize user data to a cloud service so that the user may receive professional exercise guidance at home. The exercise planning and reminding mode encourages the user to continue their initiative to exercise. Also, sharing exercise outcomes makes the exercise using the present invention more rewarding and attracts more people to participate in the exercise. Exercise amount is collected in all weathers and analyzed for the purpose of continuous exercise management.

### Embodiment III

In accordance with another aspect of the present invention, provided is an exercise health management system which includes the exercise health management terminal described in Embodiment II.

The exercise health management system can accomplish the function of a virtual personal coach and may synchronize user data to a cloud service so that the user may receive professional exercise guidance at home. The exercise planning and reminding mode encourages the user to continue their initiative to exercise. Also, sharing exercise outcomes makes the exercise using the present invention more rewarding and attracts more people to participate in the exercise. Exercise amount is collected in all weathers and analyzed for the purpose of continuous exercise management.

It is to be noted that the steps/components described in the present disclosure may be divided into further steps/components; and moreover, two or more steps/components or parts of steps/components may be combined into new steps/components according to what is required for an implementation, of which there are many, to achieve the object of the present invention.

The exercise health management method of the present invention may be embodied in hardware or firmware, or may be implemented as software or computer code that may be stored in a recording medium such as CDROM, RAM, floppy disk, hard disk or magneto-optical disk, or may be implemented as computer code that is originally stored in a remote recoding medium or a non-transient machine-readable medium and can be downloaded over a network. Therefore, the method described herein may be processed by such software stored in a recording medium using general-purpose computers, with special purpose processors, or programmable or dedicated hardware such as ASIC or FPGA. Employed computers, processors, microprocessors controllers or programmable hardware may include storage components, such as RAMs, ROMs, or flash memories that may store or receive software or computer code that implements the method described herein when being accessed and executed by the computers, processors or hardware. Furthermore, when a general-purpose computer is accessing the code to implement the method illustrated herein, execution of the code converts the general-purpose computer into a special-purpose computer with the purpose of implementing the method illustrated herein.

The invention disclosed herein has been described by means of specific embodiments. However, numerous modifications, variations and enhancements can be made thereto by those skilled in the art without departing from the spirit and scope of the invention set forth in the claims.

What is claimed is:

1. An exercise health management method, comprising the steps of:
receiving original data collected by at least one data collecting terminal;
generating an exercise solution based on the original data; and
transmitting the exercise solution to exercise equipment corresponding to the exercise solution.

2. The method of claim 1, wherein the step of generating the exercise solution based on the original data further comprises:
calculating a comprehensive exercise capability and qualified exercise objective based on the original data; and
generating an exercise solution according to the comprehensive exercise capability and qualified exercise objective.

3. The method of claim 1, wherein the original data collected by the at least one data collecting terminal comprises, but is not limited to, pace counting data collected by an intelligent watch, heart rate data collected by an intelligent chest strap, blood pressure data collected by an intelligent blood pressure monitor, water intake amount data collected by an intelligent cup, body temperature data collected by an intelligent thermometer and/or weight data collected by an intelligent body weight scale.

4. The method of claim 1, further comprising:
storing the original data and/or storing the exercise solution.

5. The method of claim 1, wherein after the step of transmitting the exercise solution to the exercise equipment corresponding to the exercise solution, the method further comprises:
receiving the original data collected by the at least one data collecting terminal; and
adjusting in real time the exercise solution based on the original data.

6. An exercise health management terminal, comprising:
a first receiving module configured to receive original data collected by at least one data collecting terminal;
a generating module configured to generate an exercise solution based on the original data received by the first receiving module; and
a transmitting module configured to transmit the exercise solution to exercise equipment corresponding to the exercise solution.

7. The terminal of claim 6, wherein the generating module comprises:
a calculating unit configured to calculate a comprehensive exercise capability and qualified exercise objective based on the original data received by the first receiving module; and
a generating unit configured to generate the exercise solution according to the comprehensive exercise capability and qualified exercise objective.

8. The terminal of claim 6, wherein the terminal further comprises a server configured to store the original data received by the first receiving module and/or store the exercise solution generated by the generating module.

9. The terminal of claim 6, wherein the terminal further comprises:
a second receiving module configured to receive in real time the original data collected by the at least one data collecting terminal; and
an adjusting module configured to adjust in real time the exercise solution based on the original data.

10. An exercise health management system comprising the exercise health management terminal of claim 7.

11. An exercise health management system comprising the exercise health management terminal of claim 8.

12. An exercise health management system comprising the exercise health management terminal of claim 9.
